(19) United States
(12) Patent Application Publication
(10) Pub. No.: US 2009/0082832 A1
(43) Pub. Date: Mar. 26, 2009

(54) Thermal Management of Implantable Medical Devices

(75) Inventors: Rafael Carbunaru, Valley Village, CA (US); Kristen N. Jaax, Santa Clarita, CA (US); Andrew Digiore, Santa Monica, CA (US); Brett Schleicher, Valencia, CA (US)

Correspondence Address: FISH & RICHARDSON, PC, P.O. BOX 1022, MINNEAPOLIS, MN, 55440-1022, US

(73) Assignee: BOSTON SCIENTIFIC NEUROMODULATION CORPORATION, Valencia, CA (US)

(21) Appl. No.: 12/237,631

(22) Filed: Sep. 25, 2008

Related U.S. Application Data
(60) Provisional application No. 60/975,111, filed on Sep. 25, 2007.

(51) Int. Cl. A61N 1/00

(52) U.S. Cl. 607/59

(57) ABSTRACT

Systems and techniques for thermal management of implantable medical devices. In one aspect, an implantable device adapted for implantation in a body includes a conductor component that conducts an electrical current in response to the body in which that implantable device is implanted being subjected to an alternating electromagnetic field and a thermal management component in thermal contact with the conductor component and configured to manage excess heat generated by the conduction of the electrical current. The thermal management component comprises a material that undergoes a phase transition at a temperature above the temperature of the body in which the implantable device is adapted to be implanted.

### CROSS-REFERENCE TO RELATED APPLICATIONS

This application claims the benefit of the priority of U.S. Provisional Application Ser. No. 60/975,111 filed on Sep. 25, 2007 and entitled “Thermal Management of Implantable Medical Devices,” the contents of which are incorporated herein by reference.

### BACKGROUND

This disclosure relates to thermal management of implantable medical devices.

When the temperature of living cells and tissues is changed, the physiological structures, compositions, and processes of the cells and tissues can also change. For example, reaction kinetics, rates of mass transport, and the stability of cellular structures are all functions of temperature. Temperature changes are often harmful and cause departures from the preferred physiological states of cells and tissues. In extreme cases, thermal necrosis (i.e., cell death due to temperature) can result.

### SUMMARY

Systems and techniques for thermal management of implantable medical devices are described. In one aspect, an implantable device that adapted for implantation in a body includes a conductor component that conducts an electrical current in response to the body in which that implantable device is implanted being subjected to an alternating electromagnetic field and a thermal management component in thermal contact with the conductor component and configured to manage excess heat generated by the conduction of the electrical current. The thermal management component comprises a material that undergoes a phase transition at a temperature between zero and ten degrees Celsius above the temperature of the body in which the implantable device is adapted to be implanted.

This and other aspects can include one or more of the following features. The conductor component can include an electrically conductive loop. The electrically conductive loop can include a conducting coil of a charging component that is configured to convert electrical or magnetic energy from outside the body in which the implantable device is adapted to be implanted. The implantable device can be a pacemaker and the conductor component can be a wire in a lead of the pacemaker. The implantable device can be an orthopedic implant. The conductor component can include a conductive linear wire in a lead, e.g., having a length of about 26 cm or about 13 cm.

In another aspect, an implantable device includes an active component configured to perform one or more medical activities, a charging component configured to convert energy from outside a body in which the implantable device is implanted into energy that can be consumed by the active component, and a thermal management component in thermal contact with the charging component and configured to manage excess heat generated by the charging component in converting energy from outside the body. The thermal management component comprises a material that undergoes a phase transition at a temperature between zero and ten degrees Celsius above the temperature of the body in which the implantable device is to be implanted.

This and other aspects can include one or more of the following features. The thermal management component can be interposed between the active component and the charging component. The thermal management component can surround the charging component. The active component can include electrical circuitry of an implantable pulse generator. The charging component can include a conducting coil. The container can be configured to restrict flow of the material of the thermal management component. For example, the container can include an elastic balloon.

The implantable device can include a thermal barrier between the active component and the charging component. The material can undergo the phase transition at a temperature between two and four degrees Celsius above the temperature of the body in which the implantable device is implanted. The charging component can include an optical component responsive to light or other electromagnetic radiation generated outside the body. The charging component can include a mechanical component responsive to mechanical impulses generated outside the body.

In another aspect, an implantable device includes a system of one or more biocompatible housings. The system can include a magnetically non-transparent biocompatible material that encloses a power storage device of an implantable pulse generator and a magnetically transparent biocompatible material that encloses a charging coil configured to convert electrical or magnetic energy from outside a body in which the implantable device is implanted in energy that can be stored at the power storage device, and a material that undergoes a phase transition at a temperature between zero and ten degrees Celsius above the temperature of a body in which the implantable device is to be implanted. The material that undergoes the phase transition can be in thermal contact with the charging coil.

This and other aspects can include one or more of the following features. The system can include a first biocompatible housing that comprises the magnetically transparent material and a second biocompatible housing that comprises the magnetically non-transparent material. The system can also include an insulated conductor that joins the first biocompatible housing and the second biocompatible housing.

The system can include a single biocompatible housing comprising a magnetically transparent portion that comprises the magnetically transparent material and a magnetically non-transparent portion that comprises the magnetically non-transparent material. The magnetically transparent portion can include a ceramic. The magnetically non-transparent portion can include a metal. The ceramic can include zirconia. The metal can include titanium. The implantable device can include a brazed seam joining the magnetically transparent portion to the magnetically non-transparent portion.

The material that undergoes the phase transition can include one or more of a clay, a paraffin, and an organic acid. The material that undergoes the phase transition can include one or more of a salt hydrate, a clathrate, and a eutectic organic or inorganic compound. The material that undergoes the phase transition can include one or more of paraffin 6106, potassium fluoride dehydrate, (KF 2H₂O), and lauric acid. In some implementations, the material that undergoes the phase transition does not conduct electricity.

The implantable device can include a container configured to restrict flow of the material of that undergoes the phase transition. The material can undergo the phase transition at a temperature between two and four degrees Celsius above the temperature of the body in which the implantable device is to be implanted.

In another aspect, an implantable device include a first hermetically-sealed housing that houses an active component configured to perform medical activities, and a second hermetically-sealed housing that houses a charging component configured to convert energy from outside a body in which the implantable device is implanted into energy that can be consumed by the active component, and a material that undergoes a phase transition at a temperature between zero and twenty degrees Celsius above the temperature of the body in which the implantable device is to be implanted.

The details of one or more implementations are set forth in the accompanying drawings and the description below. Other features and advantages will be apparent from the description and drawings, and from the claims.

### DESCRIPTION OF DRAWINGS

FIG. 1 is a schematic representation of a system in which the temperature of an implanted device is managed.

FIGS. 2-5 are schematic representations of example arrangements of components of implantable portions of the system of FIG. 1 in which excess heat generated in the conversion of energy is managed.

FIG. 6 is a schematic representation of an implantable portion of the system of FIG. 1 in which excess heat generated in the conversion of energy energy is managed.

FIGS. 7-14 are schematic representations of other implantable portions in which excess heat generated in the conversion of energy is managed.

FIG. 15 is a schematic representation of a system in which the temperature of an implanted device is managed.

FIGS. 16-19 are schematic representations of example arrangements of components of implantable portions in FIG. 15 in which excess heat generated in the conduction of an electrical current by a conductor component is managed.

FIGS. 20, 21 are schematic representations of implantable portions in which excess heat generated in the conduction of an electrical current by conductor component is managed.

Like reference symbols in the various drawings indicate like elements.

### DETAILED DESCRIPTION

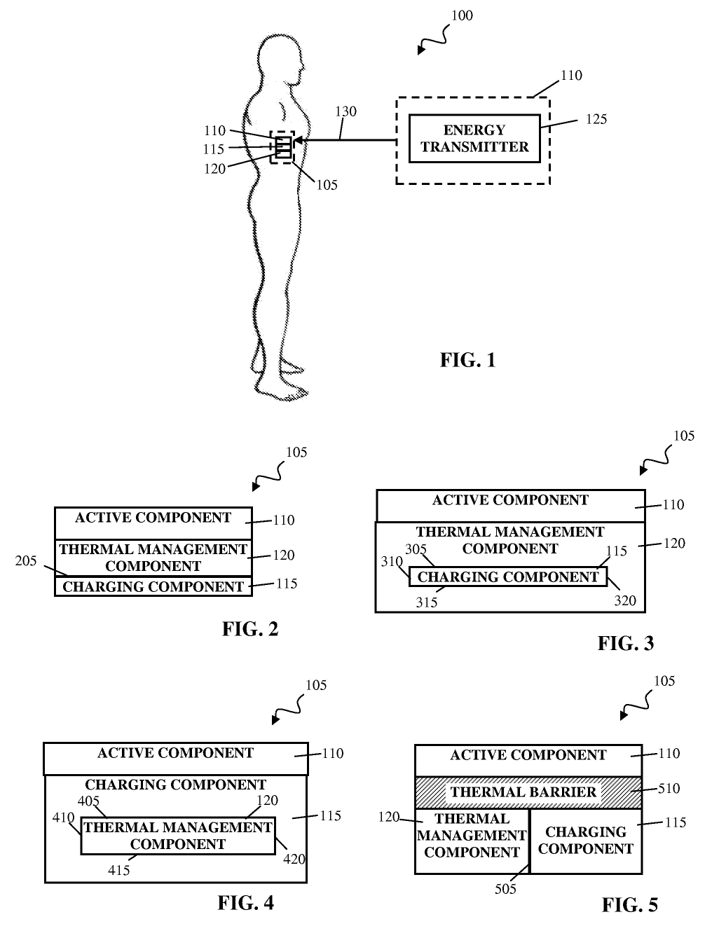

FIG. 1 is a schematic representation of a system 100 in which the temperature of an implanted device is managed. System 100 can include an implantable portion 105 and an external (i.e., extracorporeal) portion 110. Implantable portion 105 is a device that is adapted for implantation in a body. For example, implantable portion 105 can include one or more hermetically-sealed, biocompatible housings adapted to reduce the immune response and/or cell necrosis associated with the implantation of portion 105 and to isolate the interior of implantable portion 105 from the body.

Implantable portion 105 includes an active component 110, a charging component 115, and a thermal management component 120. Active component 110 is a device that performs diagnostic, therapeutic, restorative, prophylactic, monitoring, or other medical activities while implanted in the body. Active component 110 can include circuitry that manages the performance of these activities, as well as one or more power storage devices that stores energy consumed by the performance of these activities. For example, active component 110 can include electrical circuitry such as memory, timers, memory controllers, and machine logic such as logic gates, processors, ASIC's, and the like, along with power storage devices such as batteries, supercapacitors, fuel cells, mechanical energy storage devices, and the like.

The performance of activities by active component 110 consumes the energy stored at such power storage devices. Charging component 115 opposes this consumption of energy by converting energy from outside the body into energy that can be stored at such power storage devices and consumed by active component 110. For example, in some implementations, charging component 115 is a conducting coil that can respond to a magnetic or electric field generated outside a body. In other implementations, charging component 115 is an optical component that can respond to light or other electromagnetic radiation generated outside the body, such as a photovoltaic cell. In other implementations, charging component 115 is a mechanical component such as a piezoelectric resonator that can respond to acoustic oscillations or other mechanical impulses generated outside the body. The energy from charging component 115 can be stored at an energy storage device in active component 110 and consumed by active component 110.

In real devices, the conversion of energy by charging component 115 generates excess heat. For example, conducting coils have a finite resistance and heat when current flows. As another example, the conversion efficiency of photovoltaic and piezoelectric devices is not ideal.

Thermal management component 120 is a device that manages the excess heat generated by charging component 115 in converting energy from outside the body. For example, thermal management component 120 can be in thermal contact with charging component 115 to capture at least some of this excess heat. Such thermal contact can be achieved in a number of ways. For example, the thermal resistance between thermal management component 120 and charging component 115 can be relatively small due to small separation distances between components 115, 120, large contact areas between components 115, 120, active and passive thermal conduction mechanisms that transport heat between components 115, 120, and the like.

Thermal management component 120 can include one or more materials that undergo a phase transition (i.e., solid-to-liquid phase transition or liquid-to-gas phase transition) at a temperature that is above the temperature of the body in which implantable portion 105 is implanted. The transfer of excess heat from charging component 115 to thermal management component 120 allows at least some of the excess heat generated in the conversion of energy to be stored as latent heat in thermal management component 120 as it undergoes a phase transition.

In some implementations, the phase transition temperature of the materials can be in the vicinity of the temperature of the body in which implantable portion 105 is implanted. With the phase transition temperature of the materials in the vicinity of body temperature, the materials that undergo a phase transition acts as a thermal reservoir that stores excess heat and limits increases in temperature of the surrounding cells and tissues due to the excess heat. For example, in some implementations, the phase transition temperature can be between zero and twenty degrees Celsius (for example, between zero and ten or between two and four degrees Celsius) above a normal range of temperatures of the body in which implantable portion 105 is implanted. In some implementations, the phase transition temperature can be around 41 degrees Celsius for an implantable portion 105 that is designed for humans.

In some implementations, the phase transitions of the one or more materials can be associated with a relatively small volume change. The relatively small volume change can be an inherent property of the materials or can be achieved by associating the materials with a variable volume element. For example, the one or more materials can be in communication with a gas-filled balloon that can change in volume to compensate for corresponding changes in the volume of the one or more materials that undergo a phase transition.

In some implementations, the phase transition of the one or more materials spans a relatively large temperature range. For example, in some implementations, the phase transition spans between two and six degrees Celsius. This can be achieved with a single material or with a combination of materials that phase transition at different temperatures.

In some implementations, the one or more materials that undergo a phase transition can include one or more of a clay, a paraffin, a non-paraffin organic acid, a salt hydrate, a clathrate, a eutectic organic or inorganic compounds, or combinations thereof. In some implementations, the material includes paraffin 6106, potassium fluoride dehydrate, (KF 2H₂O), and/or lauric acid. In some implementations, the one or more materials that undergo a phase transition are not electrically conducting.

In some implementations, thermal management component 120 can be enclosed in a chamber or other container that restricts the flow of thermal management component 120. Examples of such containers include deformable containers (such as latex balloons and the like) that can change volume in response to changes in the volume of the one or more materials that undergo a phase transition. In some implementations, thermal management component 120 can be enclosed in a fixed volume chamber, such as a titanium or ceramic vessel.

External portion 110 of system 100 includes an energy transmitter 125 that generates an energy transmission 130. During operation, at least a portion of energy transmission 130 enters the body in which implantable portion 105 is implanted and interacts with charging component 115. Charging component 115 responds to the interaction with energy transmission 130 by converting energy transmission 130 into energy that can be stored at power storage devices and consumed by active component 110. For example, in some implementations, energy transmitter 125 is a biased coil that generates a magnetic or electric field transmission 130 that interacts with a conducting coil charging component 115. In other implementations, energy transmitter 125 is a laser, a bulb, or other light source that generates an electromagnetic radiation transmission 130 (including infra-red in a particular implementation) that interacts with a photovoltaic charging component 115. In other implementations, energy transmitter 125 is a piezoelectric crystal or other pressure source that generates a mechanical impulse transmission 130 that interacts with a mechanical charging component 125.

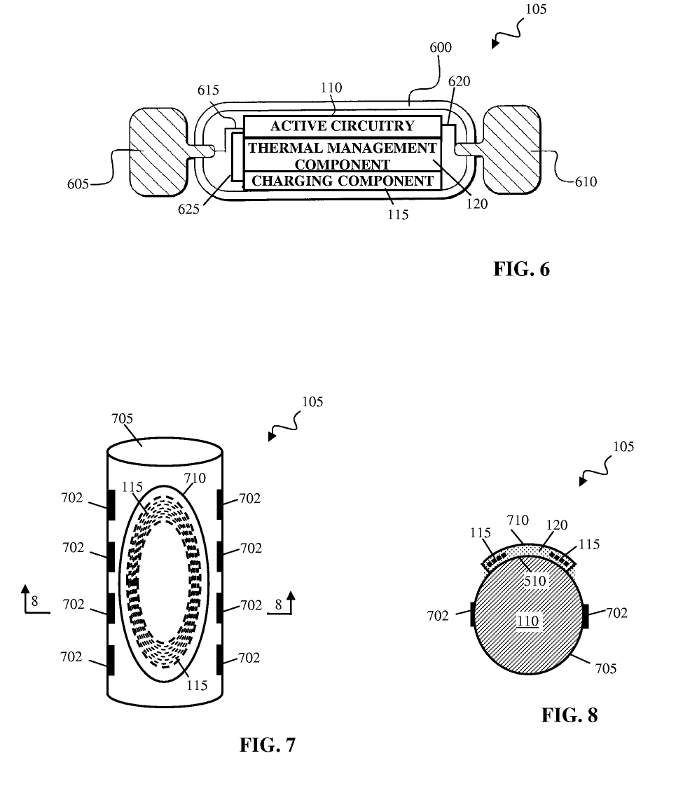

FIGS. 2-5 are schematic representations of example arrangements of components 110, 115, 120 in various implantable portions 105 in which excess heat generated in the conversion of energy by charging component 115 is managed.

In FIG. 2, thermal management component 120 is interposed between active component 110 and charging component 115. Thermal management component 120 is in thermal contact with charging component 115 across a face 205. In some implementations, elements of active component 110 that are relatively resistant to temperature increases, such as the board of a flexible-board circuit, can be positioned closer to thermal management component 120 than temperature sensitive elements in active component 110. In some implementation, elements that tend to generate excess heat during operation can be positioned closer to thermal management component 120. Thus, thermal management component 120 can also manage excess heat generated by “hot spots” of a flexible-board circuit or by other elements in active component 110.

In FIG. 3, charging component 115 is surrounded by thermal management component 120. Active component 110 is disposed off to one or more sides of thermal management component 120. Thermal management component 120 is in thermal contact with charging component 115 across a collection of faces 305, 310, 315, 320. Thus, thermal management component 120 can be in thermal contact across more than one face of charging component 115, up to and including all faces of charging component 115.

In FIG. 4, thermal management component 120 is enclosed within charging component 115. Active component 110 is disposed off to one or more sides of charging component 115. Thermal management component 120 is in thermal contact with charging component 115 across a collection of faces 405, 410, 415, 420. Thus, charging component 115 can be in thermal contact across more than one face of thermal management component 120, up to and including all faces of thermal management component 120.

In FIG. 5, thermal management component 120 is in thermal contact with charging component 115 across at least one face 505. One or both of thermal management component 120 and charging component 115 is thermally isolated from active component 110 by a thermal barrier 510. Thermal barrier 510 thermally isolates one or both of thermal management component 120 and charging component 115 from active component 110. The thermal isolation provided by thermal barrier 510 can hinder heat transport from charging component 115 to active component 110 and/or from thermal management component 120 to active component 110. For example, thermal barrier 510 can raise the thermal resistance between charging component 115 and active component 110 above the thermal resistance between charging component 115 and a body in which implantable portion 105 is implanted.

The elevated thermal resistance of thermal barrier 510 can be achieved in a number of different ways. For example, thermal barrier 510 can passively or actively manage the flow of heat. For example, thermal barrier 510 can be made from a material of a relatively low thermal conductivity. As another example, thermal barrier 510 can be made relatively thick. As yet another example, the area of thermal barrier 510 can be dimensioned to be relatively small. As a still further example, an active fluid flow device or a passive thermal conductor component can conduct heat away from thermal barrier 510. Combinations of these and other approaches can also be used.

In some implementations, components 110, 115, 120 can be housed in a single biocompatible housing. In other implementations, components 110, 115, 120 can be housed in multiple biocompatible housings, as discussed further below. Additional arrangements of components 110, 115, 120 are also available.

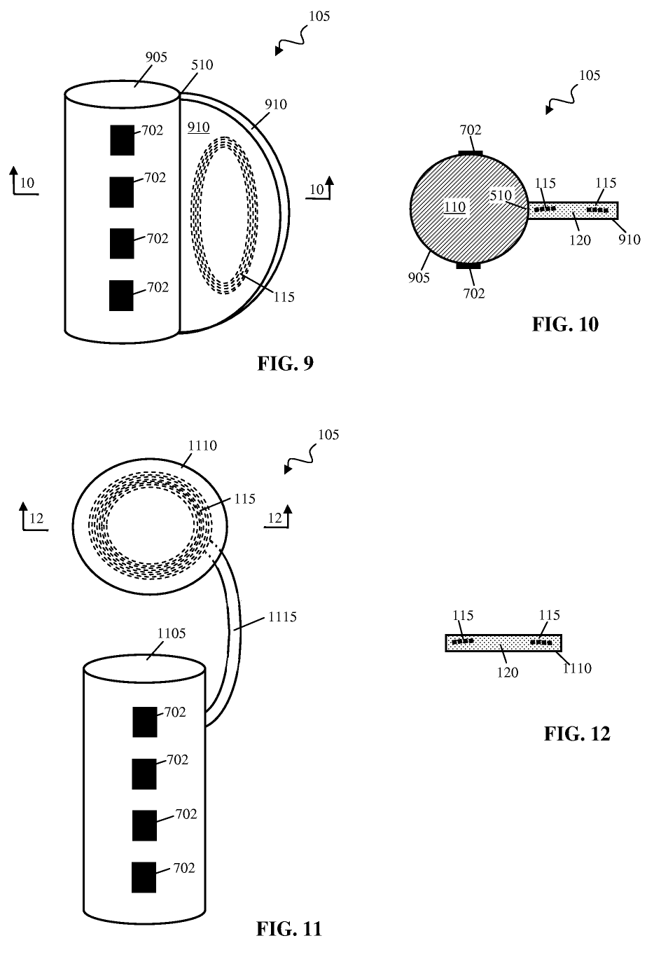

FIG. 6 is a schematic representation of an implantable portion 105 in which excess heat generated in the conversion of energy by charging component 115 is managed. In the illustrated implementation, implantable portion 105 is an implantable pulse generator (IPG) that is configured to deliver electrical pulses to tissue. Implantable portion 105 includes a single biocompatible housing 600 that houses active component 110, charging component 115, and thermal management component 120. Thermal management component 120 is disposed between active component 110 and charging component 115 in housing 600. Housing 600 can be formed of a biocompatible material, such as a magnetically transparent biocompatible ceramic or polymer. Examples of such biocompatible materials include ceramics such as zirconia, alumina, and the like and polymers such as silicone, urethane, and the like.

Active component 110 includes electrical circuitry that controls the delivery of electrical pulses to one or more electrodes 605, 610 that traverse biocompatible housing 600. Electrodes 605, 610 can include one or more cathodes to electrically excite the depolarization of a nerve and/or muscle tissue and/or one or more anodes to hyperpolarize nerve and/or muscle tissue. Electrodes 605, 610 are joined to the electrical circuitry of active component 110 by conductors 615, 620. Charging component 115 is joined to the electrical circuitry of active component 110 by one or more conductors 625.

In some implementations, an IPG implantable portion 105 can be a microstimulator that is dimensioned to be implanted in a body through the cannula of a surgical tool, such as an endoscopic or a laparoscopic device, for performing closed surgical procedures. One example of such a microstimulator is the BION neurostimulator developed by Advanced Bionics (Sylmar, Calif.). The BION can be a leadless neurostimulator that is formed from an ovoid cylinder that is about 30 millimeters in length and has a width of about 7.5 millimeters and a thickness of about 4.3 mm.

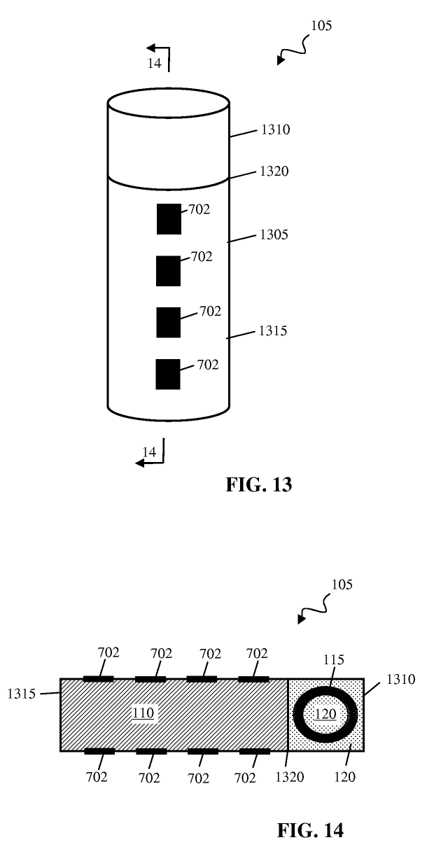

This form factor allows the BION to be implanted with relative ease and rapidity, e.g., via endoscopic or laparoscopic techniques. In some implementations, the BION consists of eight electrodes that are disposed in groups of four on opposite sides of a biocompatible casing, as shown in FIG. 7. In other implementations, one or more electrodes can be disposed on a lead attached to the biocompatible casing. Various features and details associated with the manufacture, operation, and use of BION implantable microstimulators are described in U.S. Pat. No. 5,193,539, U.S. Pat. No. 5,193,540, U.S. Pat. No. 5,312,439, U.S. Pat. No. 5,324,316, U.S. Pat. No. 5,405,367, U.S. Pat. No. 6,051,017, PCT Publication No. WO 98/37926, PCT Publication No. WO 98/43700, PCT Publication No. WO 98/43701, and the publication entitled “Micromodular Implants to Provide September Electrical Stimulation of Paralyzed Muscles and Limbs” by Cameron, et al. in IEEE Transactions on Biomedical Engineering, Vol. 44, No. 9, pages 781 790 (1997), the contents of all of which are incorporated herein by reference.

In such microstimulators, charging component 115 can include a conducting coil that can respond to a magnetic or electric field generated outside a body. In particular, an external magnetic or electric field can drive electrons in such a coil and the motion of these electrons can be converted and stored in a battery or other storage device for consumption by active component 110.

FIGS. 7 and 8 are schematic representations of another IPG implantable portion 105 in which excess heat generated in the conversion of energy by charging component 115 is managed. Implantable portion 105 includes a collection of electrodes 702 and a pair of biocompatible housings 705, 710. Electrodes 702 can act as cathodes to electrically excite the depolarization of a nerve and/or muscle tissue and/or anodes to hyperpolarize nerve and/or muscle tissue. In some implementations, electrodes 702 can be disposed in groups of four on opposite sides of housing 705.

Housing 705 houses the electrical circuitry of active component 110 and can be formed, e.g., of a biocompatible metal, polymer, or ceramic. Housing 710 houses charging component 115 and thermal management component 120 and can be formed of a magnetically transparent material such as, e.g., biocompatible ceramic or polymer. In some implementations, housing 710 can have a variable volume in that housing 710 expands and contracts in response to any temperature-driven volume changes in charging component 115 and/or thermal management component 120. Such volume variability can be achieved, e.g., when housing 710 is formed of a polymeric material that is relatively elastic. In other implementations, housing 710 can house a void volume and an internal variable volume container that not only restricts the flow of thermal management component but also expands and contracts in response to any temperature-driven volume changes in charging component 115 and/or thermal management component 120.

Contacting faces of housings 705, 710 can act as a thermal barrier 510 that thermally isolates thermal management component 120 and charging component 115 from active component 110. In some implementations, the thermal resistance between charging component 115 and active component 110 can be raised above the thermal resistance between charging component 115 and the body.

FIGS. 9 and 10 are schematic representations of another IPG implantable portion 105 in which excess heat generated in the conversion of energy by charging component 115 is managed. Implantable portion 105 includes a collection of electrodes 702 and a pair of biocompatible housings 905, 910.

Housing 905 houses the electrical circuitry of active component 110 and can be formed, e.g., of a biocompatible metal, polymer, or ceramic. Housing 910 is a flapped element that houses thermal management component 120 and a conducting coil charging component 115 so that the windings of the coil follow a path around normals to the larger faces of flapped housing 910. The flap of housing 910 is generally positioned to extend outwardly away from housing 905. The area of contact between housings 905, 910 can act as a thermal barrier 510 that thermally isolates thermal management component 120 and charging component 115 from active component 110. In some implementations, the thermal resistance between charging component 115 and active component 110 can be raised above the thermal resistance between charging component 115 and the body. For example, the area of barrier 510 can be relatively small in comparison to the total area of housing 910.

Housing 910 can be formed of a magnetically transparent material such as, e.g., biocompatible ceramic or polymer. In some implementations, housing 910 can have a variable volume in that housing 910 expands and contracts in response to any temperature-driven volume changes in charging component 115 and/or thermal management component 120. Such volume variability can be achieved, e.g., when housing 910 is formed of a polymeric material that is relatively elastic. In other implementations, housing 910 can house a void volume and an internal variable volume container that not only restricts the flow of thermal management component but also expands and contracts in response to any temperature-driven volume changes in charging component 115 and/or thermal management component 120.

FIGS. 11 and 12 are schematic representations of another IPG implantable portion 105 in which excess heat generated in the conversion of energy by charging component 115 is managed. Implantable portion 105 includes a collection of electrodes 702 and a pair of biocompatible housings 1105, 1110.

Housing 1105 houses the electrical circuitry of active component 110 and can be formed, e.g., of a biocompatible metal, polymer, or ceramic. Housing 1110 houses thermal management component 120 and a conducting coil charging component 115. Housing 1105, 1110 are joined by an insulated conductor 1115. Insulated conductor 1115 includes wires or other components that allow the active component 110 to access the energy from charging component 115. The separation between active component 110 and charging component 115 spanned by insulated conductor 1115 acts as a thermal barrier and thermally isolates charging component 115 from active component 110, including any circuitry and power storage device(s) therein.

In some implementations, insulated conductor 1115 can be flexible in that relative motion and/or rotation between housings 1105, 1110 is achieved when implantable portion 105 is subject to physiological-scale stresses associated with implantation. In other implementations, insulated conductor 1205 can be stiff in that housings 1105, 1110 are held in a fixed positional relationship when implantable portion 105 is subject to physiological-scale stresses associated with implantation. In either case, the outer surface of insulated conductor 1115 can be made of a biocompatible insulator and adapted to reduce the immune response and/or cell necrosis associated with implantation.

Housing 1110 can be formed of a magnetically transparent material such as, e.g., biocompatible ceramic or polymer. In some implementations, housing 1110 can have a variable volume in that housing 1110 expands and contracts in response to any temperature-driven volume changes in charging component 115 and/or thermal management component 120. Such volume variability can be achieved, e.g., when housing 1110 is formed of a polymeric material that is relatively elastic. In other implementations, housing 1110 can house a void volume and an internal variable volume container that not only restricts the flow of thermal management component but also expands and contracts in response to any temperature-driven volume changes in the one or more materials that undergo a phase transition in charging component 115 and/or thermal management component 120.

FIGS. 13 and 14 are schematic representations of another IPG implantable portion 105 in which excess heat generated in the conversion of energy by charging component 115 is managed. Implantable portion 105 includes a collection of electrodes 702 and a single biocompatible housing 1305.

Housing 1305 includes a magnetically transparent portion 1310 and a magnetically non-transparent portion 1315. Magnetically transparent portion 1310 is formed from a magnetically transparent material that does not substantially hinder the through transmission of a magnetic or electric field. For example, magnetically transparent portion 1310 can be formed from a ceramic such as zirconia or alumina and/or from a polymeric material. Magnetically non-transparent portion 1315 is formed from a material that is not magnetically transparent in that it hinders the through transmission of a magnetic or electric field. For example, magnetically non-transparent portion 1315 can be formed from metal (e.g., titanium), glass, ceramic, plastic, as well as alloys, cermets, reaction products, composites, and mixtures comprising at least one of the foregoing, in combination with magnetically impermeable fillers where needed to attain the desired relative magnetic permeability. Examples of such fillers include nickel, iron, cobalt, manganese, gadolinium, dysprosium, oxides such as iron oxide, ferrites (such as LiZn (lithium zinc) ferrites, MgMnZn (magnesium manganese zinc) ferrites, and the like), cermets, alloys, laminates, and combinations comprising the foregoing materials.

Magnetically transparent portion 1310 and magnetically non-transparent portion 1315 can be joined at a seam 1320. Seam 1320 can hermetically seal implantable portion 105. In some implementations, seam 1320 can be formed from a solder or brazing material.

Magnetically non-transparent portion 1315 of housing 1305 houses at least a portion of active component 110, such as a battery or other power storage device. Magnetically transparent portion 1310 of housing 1305 houses conducting coil charging component 115 and at least a portion of thermal management component 120. In some implementations, magnetically transparent portion 1310 can also house portions of active component 110. For example, magnetically transparent portion 1310 can house all of the electrical circuitry in active component 110. As another example, magnetically transparent portion 1310 can house some subset of the electrical circuitry in active component 110, such as the electrical components that generate excess heat during operation.

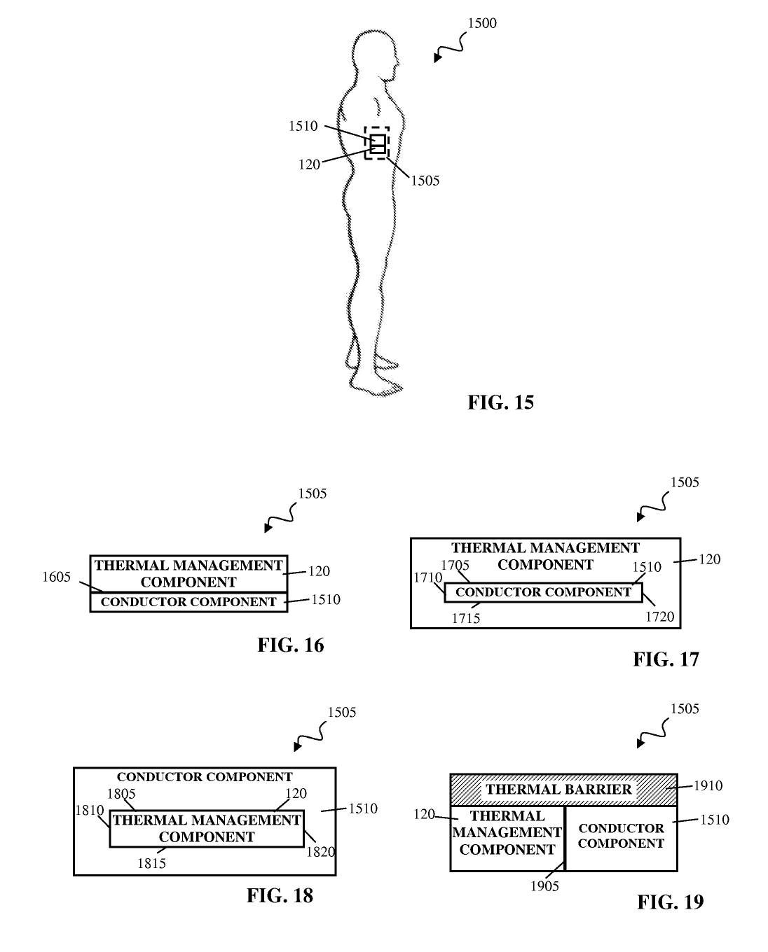

FIG. 15 is a schematic representation of another system 1500 in which the temperature of an implanted device is managed. System 1500 can include an implantable portion 1505 that is adapted for implantation in a body. For example, implantable portion 1505 can include one or more hermetically-sealed, biocompatible housings adapted to reduce the immune response and/or cell necrosis associated with the implantation of portion 1505 and to isolate the interior of implantable portion 1505 from the body. Implantable portion 1505 can be, e.g., an active implantable medical device (i.e., a medical device powered by a source of energy other than the human body or gravity), a semi-active implantable medical device (i.e., a medical device powered from outside the body), or a passive implantable medical device (i.e., a medical device that is not powered at all). For example, implantable portion 1505 can be a cochlear implant, a bone fusion stimulator, a Foley catheter, an infusion pump, a deep brain stimulator, a spinal cord stimulator, a nerve stimulator, an orthopedic implant, or the like.

Implantable portion 1505 includes a conductor component 1510 and a thermal management component 120. Conductor component 1510 is a component of implantable portion 1505 that conducts electricity and can be made, e.g., from metal. Conductor component 1510 can play an number of different roles in implantable portion 1505. For example, conductor component 1510 can act as the electrically conductive wire or electrical shielding in a lead that conducts electrical signals, e.g., in active devices such as pacemakers. As another example, conductor component 1510 can act as all or a portion of a housing that isolates the interior of implantable portion 1505 from the body. As yet another example, conductor component 1510 can act to provide or supplement the mechanical integrity of the body in which implantable portion 1505 is implanted. For example, conductor component 1510 can be a component of a orthopedic device such as, e.g., a cervical or other fixation device.

Conductor component 1510 can conduct an electrical current in response to the body in which implantable portion 1505 is implanted being subjected to an alternating electromagnetic field. For example, in response to the body in which implantable portion 1505 is implanted being subject to a radio frequency (RF) field (e.g., from an magnetic resonance imaging (MRI) device), conductor component 1510 can conduct electrical current between electrical potentials in the body that are induced by the RF field.

As another example, conductor component 1510 can interact with the oscillating magnetic component and/or the oscillating electric component of the alternating electromagnetic field itself and conduct electricity. For example, the oscillating magnetic component of an RF field can induce eddy currents in conductor component 1510. As another example, the oscillating electric component of an RF field can support resonant waves and/or resonant conducting loops. Thus, in some implementations, conductor component 1510 can include one or more conductive loops. For example, conductor component 1510 can include a conducting coil, such as found in some implementations of charging component 115.

As another example, in some implementations, conductor component 1510 can be dimensioned to support resonant waves under imaging conditions that are characteristic of MRI imaging. By way of example, if conductor component 1510 is a linear conductor such as the conductive wire in a lead of a pacemaker, conductor component 1510 can have a length that is in the vicinity of one-half the wavelength of an RF field that is characteristic of magnetic resonance imaging. For example, a linear conductor component 1510 can have a length of about 26 cm and/or about 13 cm, which are in the vicinity of one-half the wavelengths of the RF fields characteristic of magnetic resonance imaging at 64 MHz/1.5 Tesla and 128 MHz/3.0 Tesla, respectively.

The conduction of an electrical current by conductor component 1510—during MRI imaging or otherwise—can generate excess heat. For example, resistive (e.g., ohmic) heating can occur due to the conduction of electricity within conductor component 1510. As another example, resistive heating can occur due to the conduction of electricity across an interface between conductor component 1510 and another element, such as the body.

Thermal management component 120 manages the excess heat generated by conductor component 1510 in conducting electricity, e.g., during MRI imaging. Thermal management component 120 can be in thermal contact with conductor component 1510 to capture at least some of this excess heat. Such thermal contact can be achieved in a number of ways. For example, the thermal resistance between thermal management component 120 and conductor component 1510 can be relatively small due to small separation distances between components 1510, 120, large contact areas between components 1510, 120, active and passive thermal conduction mechanisms that transport heat between components 1510, 120, and the like.

Thermal management component 120 can include one or more materials that undergo a phase transition (i.e., solid-to-liquid phase transition or liquid-to-gas phase transition) at a temperature that is above the temperature of the body in which implantable portion 1505 is implanted. The transfer of excess heat from conductor component 1510 to thermal management component 120 allows at least some of the excess heat generated in the conversion of energy to be stored as latent heat in thermal management component 120 as it undergoes a phase transition.

In some implementations, the phase transition temperature of the materials can be in the vicinity of the temperature of the body in which implantable portion 1505 is implanted. With the phase transition temperature of the materials in the vicinity of body temperature, the materials that undergo a phase transition acts as a thermal reservoir that stores excess heat and limits increases in temperature of the surrounding cells and tissues due to the excess heat. For example, in some implementations, the phase transition temperature can be between zero and twenty degrees Celsius (for example, between zero and ten or between two and four degrees Celsius) above a normal range of temperatures of the body in which implantable portion 105 is implanted. In some implementations, the phase transition temperature can be around 41 degrees Celsius for an implantable portion 1505 that is designed for humans.

In some implementations, the phase transitions of the one or more materials can be associated with a relatively small volume change. The relatively small volume change can be an inherent property of the materials or can be achieved by associating the materials with a variable volume element. For example, the one or more materials can be in communication with a gas-filled balloon that can change in volume to compensate for corresponding changes in the volume of the one or more materials that undergo a phase transition.

In some implementations, the phase transition of the one or more materials spans a relatively large temperature range. For example, in some implementations, the phase transition spans between two and six degrees Celsius. This can be achieved with a single material or with a combination of materials that phase transition at different temperatures.

In some implementations, the one or more materials that undergo a phase transition can include one or more of a clay, a paraffin, a non-paraffin organic acid, a salt hydrate, a clathrate, a eutectic organic or inorganic compounds, or combinations thereof. In some implementations, the material includes paraffin 6106, potassium fluoride dehydrate, (KF 2H₂O), and/or lauric acid. In some implementations, the one or more materials that undergo a phase transition are not electrically conducting.

In some implementations, thermal management component 120 can be enclosed in a chamber or other container that restricts the flow of thermal management component 120. Examples of such containers include deformable containers (such as latex balloons and the like) that can change volume in response to changes in the volume of the one or more materials that undergo a phase transition. In some implementations, thermal management component 120 can be enclosed in a fixed volume chamber, such as a titanium or ceramic vessel.

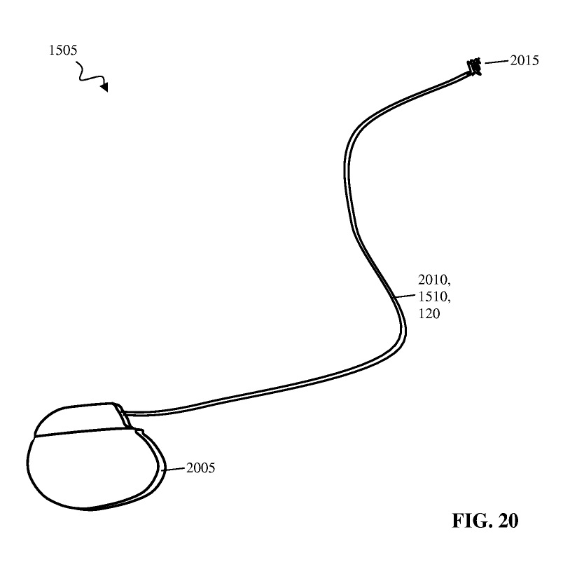

FIGS. 16-19 are schematic representations of example arrangements of components 1510, 120 in various implantable portions 1505 in which excess heat generated in the conduction of an electrical current by conductor component 1510 is managed.

In FIG. 16, thermal management component 120 is in direct thermal and physical contact with conductor component 1510 across a face 1605.

In FIG. 17, conductor component 1510 is surrounded by thermal management component 120. Thermal management component 120 is in thermal contact with conductor component 1510 across a collection of faces 1705, 1710, 1715, 1720. Thus, thermal management component 120 can be in thermal contact across more than one face of conductor component 1510, up to and including all faces of conductor component 1510.

In FIG. 18, thermal management component 120 is enclosed within conductor component 1510. Thermal management component 120 is in thermal contact with conductor component 1510 across a collection of faces 1805, 1810, 1815, 1820. Thus, conductor component 1510 can be in thermal contact across more than one face of thermal management component 120, up to and including all faces of thermal management component 120.

In FIG. 19, thermal management component 120 is in thermal contact with conductor component 1510 across at least one face 1905. One or both of thermal management component 120 and conductor component 1510 is thermally isolated from, e.g., the remainder of implantable portion 1505 or the body in which implantable portion 1505 is implanted, by a thermal barrier 1910. Thermal barrier 1910 thermally isolates thermal management component 120 and conductor component 1510. The thermal isolation provided by thermal barrier 1910 can hinder heat transport from conductor component 1510. For example, thermal barrier 1910 can raise the thermal resistance between conductor component 1510 and the body in which implantable portion 1510 is implanted above the thermal resistance between conductor component 1510 and thermal management component 120.

The elevated thermal resistance of thermal barrier 1910 can be achieved in a number of different ways. For example, thermal barrier 1910 can passively or actively manage the flow of heat. For example, thermal barrier 1910 can be made from a material of a relatively low thermal conductivity. As another example, thermal barrier 1910 can be made relatively thick. As yet another example, the area of thermal barrier 1910 can be dimensioned to be relatively small. As a still further example, an active fluid flow device or a passive thermal conductor component can conduct heat away from thermal barrier 1910. Combinations of these and other approaches can also be used.

Additional arrangements of components 1510, 120 are also available.

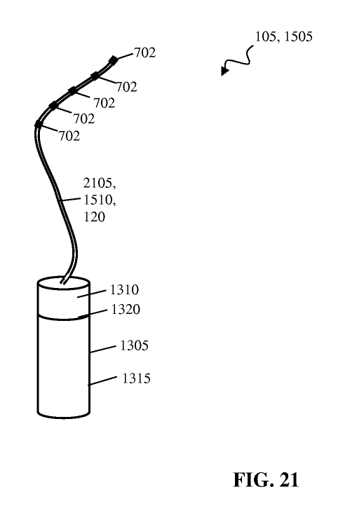

FIG. 20 is a schematic representation of an implantable portion 1505 in which excess heat generated in the conduction of an electrical current by conductor component 1510 is managed. In the illustrated implementation, implantable portion 1505 is a pacemaker that is configured to deliver electrical pulses to the heart. Implantable portion 1505 includes a casing 2005, a lead 2010, and an electrode 2015. Lead 2010 includes a biocompatible, insulating sheath that surrounds one or more conducting wires. Such a conducting wire in lead 2010 is a conductor component 1510 and can be arranged in thermal contact with thermal management component 120 to manage heat generated in the conduction of an electrical current as described, e.g., in FIGS. 16-19. The electrical current that is managed can be conducted, e.g., during MRI imaging.

FIG. 21 is a schematic representation of an implantable portion 1505 in which excess heat generated in the conduction of an electrical current by conductor component 1510 and in the conversion of energy by charging component 115 is managed. In the illustrated implementation, implantable portion 1505 is an IPG implantable portion 105.

The illustrated implementation of IPG implantable portion 105 includes a single biocompatible housing 1305, a lead 2105, and a collection of ring and tip electrodes 702 arrayed along lead 2105. Lead 2105 includes a biocompatible, insulating sheath that surrounds a collection of conducting wires that make electrical contact to electrodes 702. Such conducting wires in lead 2105 are conductor components 1510 and can be arranged in thermal contact with a first thermal management component 120 to manage heat generated in the conduction of an electrical current as described, e.g., in FIGS. 16-19. The electrical current that is managed can be conducted, e.g., during MRI imaging.

Magnetically transparent portion 1310 of housing 1305 houses conducting coil charging component 115 and a second thermal management component 120. Charging component 115 can be arranged in thermal contact with the second thermal management component 120 to manage heat generated in the conversion of energy by charging component 115 as described, e.g., in FIGS. 2-5.

A number of implementations have been described. Nevertheless, it will be understood that various modifications may be made. For example, thermal management component 120 may also include active or passive elements that redistribute heat in implantable portions 105, 1505. For example, a material that undergoes a phase transition can flow from hot spots in implantable portion 105, 1505 to cool spots to redistribute heat in implantable portion 105, 1505.

Accordingly, other implementations are within the scope of the following claims.

What is claimed is:

1. An implantable device adapted for implantation in a body, comprising:
a conductor component that conducts an electrical current in response to the body in which that implantable device is implanted being subjected to an alternating electromagnetic field; and
a thermal management component in thermal contact with the conductor component and configured to manage excess heat generated by the conduction of the electrical current;
wherein the thermal management component comprises a material that undergoes a phase transition at a temperature between zero and ten degrees Celsius above the temperature of the body in which the implantable device is adapted to be implanted.

2. The implantable device of claim 1, wherein the conductor component comprises an electrically conductive loop.

3. The implantable device of claim 2, wherein the electrically conductive loop comprises a conducting coil of a charging component that is configured to convert electrical or magnetic energy from outside the body in which the implantable device is adapted to be implanted.

4. The implantable device of claim 1, wherein:
the implantable device comprises a pacemaker; and
the conductor component comprises a wire in a lead of the pacemaker.

5. The implantable device of claim 1, wherein:
the implantable device comprises a neurostimulator; and
the conductor component comprises a wire in a lead of the neurostimulator.

6. The implantable device of claim 1, wherein the implantable device comprises an orthopedic implant.

7. The implantable device of claim 1, wherein the conductor component comprises a conductive linear wire in a lead.

8. The implantable device of claim 1, wherein the conductive linear wire has a length of about 26 cm or about 13 cm.

9. The implantable device of claim 1, wherein the implantable device further comprises:
an active component configured to perform one or more medical activities; and
a charging component that comprises the conductor component and is configured to convert energy from outside a body in which the implantable device is implanted into energy that can consumed by the active component,
wherein
the conversion of energy includes the conduction of the electrical current by the conductor component, and
the thermal management component is in thermal contact with the conductor component of the charging component and is configured to manage excess heat generated in the conversion of energy from outside the body.

10. The implantable device of claim 9, wherein the thermal management component is interposed between the active component and the charging component.

11. The implantable device of claim 9, wherein the thermal management component surrounds the charging component.

12. The implantable device of claim 9, wherein the active component comprises electrical circuitry of an implantable pulse generator.

13. The implantable device of claim 9, wherein the charging component comprises a conducting coil.

14. The implantable device of claim 9, further comprising a thermal barrier between the active component and the charging component.

15. The implantable device of claim 1, further comprising a container configured to restrict flow of the material of the thermal management component.

16. The implantable device of claim 15, wherein the container comprises an elastic balloon.

17. The implantable device of claim 1, wherein the material undergoes the phase transition at a temperature between two and four degrees Celsius above the temperature of the body in which the implantable device is adapted for implantation.

18. The implantable device of claim 1, further comprising:
a system of one or more biocompatible housings, wherein at least one of the housings in the system comprises:
a magnetically non-transparent biocompatible material that encloses a power storage device of an implantable pulse generator; and
a magnetically transparent biocompatible material that encloses the conductor component and the thermal management component, wherein the conductor component comprises a charging coil configured to convert electrical or magnetic energy from outside a body in which the implantable device is implanted into energy that can be stored at the power storage device and the thermal management component is in thermal contact with the charging coil.

19. The implantable device of claim 18, wherein the system comprises:
a first biocompatible housing that comprises the magnetically transparent material; and
a second biocompatible housing that comprises the magnetically non-transparent material.

20. The implantable device of claim 19, wherein the system further comprises an insulated conductor that joins the first biocompatible housing and the second biocompatible housing.

21. The implantable device of claim 18, wherein the system comprises a single biocompatible housing comprising a magnetically transparent portion that comprises the magnetically transparent material and a magnetically non-transparent portion that comprises the magnetically non-transparent material.

22. The implantable device of claim 21, wherein:
the magnetically transparent portion comprises a ceramic;
the magnetically non-transparent portion comprises a metal.

23. The implantable device of claim 22, wherein:
the ceramic comprises zirconia; and
the metal comprises titanium.

24. The implantable device of claim 21, further comprising a brazed seam joining the magnetically transparent portion to the magnetically non-transparent portion.

25. The implantable device of claim 1, wherein the material that undergoes the phase transition comprises one or more of a clay, a paraffin, and an organic acid.

26. The implantable device of claim 1, wherein the material that undergoes the phase transition comprises one or more of a salt hydrate, a clathrate, and a eutectic organic or inorganic compound.

27. The implantable device of claim 1, wherein the material that undergoes the phase transition comprises one or more of paraffin 6106, potassium fluoride dehydrate, (KF 2H₂O), and lauric acid.

28. The implantable device of claim 1, wherein the material that undergoes the phase transition does not conduct electricity.

29. The implantable device of claim 1, wherein the material undergoes the phase transition at a temperature between two and four degrees Celsius above the temperature of the body in which the implantable device is to be implanted.

30. An implantable device comprising:
a first hermetically-sealed housing that houses an active component configured to perform medical activities;
a second hermetically-sealed housing that houses
a charging component configured to convert energy from outside a body in which the implantable device is implanted into energy that can be consumed by the active component, and
a material that undergoes a phase transition at a temperature between zero and twenty degrees Celsius above the temperature of the body in which the implantable device is to be implanted; and
an insulated conductor configured to conduct energy from the second hermetically-sealed housing to the active component housed in the first hermetically-sealed housing.

31. An implantable device comprising:
an active component configured to perform one or more medical activities;
a charging component configured to convert energy from outside a body in which the implantable device is implanted into potential energy; and
a thermal management component in thermal contact with the charging component and configured to manage excess heat generated by the charging component in converting energy from outside the body into potential energy,
wherein the thermal management component comprises a material that undergoes a phase transition at a temperature between zero and ten degrees above the temperature of the body in which the implantable device is to be implanted.

32. The implantable device of claim 31, wherein the charging component comprises an optical component responsive to light or other electromagnetic radiation generated outside the body.

33. The implantable device of claim 31, wherein the charging component comprises a mechanical component responsive to mechanical impulses generated outside the body.
